(19) United States
(12) Patent Application Publication
(10) Pub. No.: US 2019/0000501 A1
(43) Pub. Date: Jan. 3, 2019

(54) SYSTEMS, DEVICES AND METHODS FOR ACCESSING A BODY

(71) Applicants: Bryan Nowroozi, Laguna Niguel, CA (US); Saheel Sutaria, El Cerrito, CA (US); Daniel Burnett, San Francisco, CA (US)

(72) Inventors: Bryan Nowroozi, Laguna Niguel, CA (US); Saheel Sutaria, El Cerrito, CA (US); Daniel Burnett, San Francisco, CA (US)

(21) Appl. No.: 16/027,064

(22) Filed: Jul. 3, 2018

Related U.S. Application Data
(60) Provisional application No. 62/528,388, filed on Jul. 3, 2017.
(60) Provisional application No. 62/652,448, filed on Apr. 4, 2018.

(51) Int. Cl. A61B 17/34; A61B 18/14

(52) U.S. Cl. CPC A61B 17/3403; A61B 18/1487; A61B 2018/00755; A61B 2017/3456; A61B 2017/347; A61B 17/3423

(57) ABSTRACT

Body access device variations are described herein which may comprise a cannula configured for insertion into a body, an electrode at or near a distal end of the cannula, where the electrode is positioned to contact tissue adjacent to the distal end of the cannula during advancement into the body. The device may include another electrode, a controller in communication with the first electrode via a first lead and in communication with the second electrode via a second lead, where the first lead runs through an inner lumen of the cannula and where the device is configured to detect an impedance or a conductance of the tissue between the first electrode and the second electrode, and where the controller is configured to receive one or more signals from the tissue adjacent to the distal end of the cannula and detect a change in property of the tissue as the cannula is advanced, and where the controller is further configured to determine a position of the distal end of the cannula during advancement into the body.

### CROSS-REFERENCE TO RELATED APPLICATIONS

This application claims the benefit of priority to U.S. Provisional Application No. 62/528,388 filed Jul. 3, 2017 and U.S. Provisional Application No. 62/652,448 filed Apr. 4, 2018, each of which is incorporated herein by reference in its entirety.

### FIELD

Embodiments disclosed herein relate to devices and methods for accessing body cavities or other body areas.

### INCORPORATION BY REFERENCE

All publications and patent applications mentioned in this specification are herein incorporated by reference to the same extent as if each such individual publication or patent application were specifically and individually indicated to be so incorporated by reference.

### BACKGROUND

Certain medical procedures, including laparoscopy, endoscopy, tissue sampling, biopsy, tracheotomy, vascular access, central nervous system access, lung access, amniotic access, tumor access, natural body lumen access, etc., require fairly precise access to the body. Current trocar, cannula, needle and other types of access systems are often “blind”, or require auxiliary visualization, such as fluoroscopy, X-ray, ultrasound imaging etc.

An access device is needed which can alert the user when it is in the desired location in the body.

### SUMMARY

The conductive access devices and methods disclosed herein include conductive areas which can determine the conductivity and/or impedance of tissue as the device is moved through the tissue and placed. Certain changes in conductivity/impedance indicate when the access device is placed in its desired location. The conductive/impedance properties of the body tissues may be inherent, or may be altered by the device, or otherwise. For example, a blunt access device may blanch tissue as the tissue is spread by the device, creating changes in tissue conductivity/impedance. Another example is an access device which includes cautery or ablation capabilities. The cauterization or ablation of the tissue may change the conductivity/impedance of the tissue.

One embodiment of the access device includes an apparatus for accessing a portion of a body including a cannula configured for insertion into the body, a first electrode at or near a distal end of the cannula, where the first electrode is positioned to contact tissue adjacent to the distal end of the cannula during advancement into the body, a second electrode, a controller in communication with the first electrode via a first lead and in communication with the second electrode via a second lead, where the first lead runs through an inner lumen of the cannula and where the apparatus is configured to detect an impedance or a conductance of the tissue between the first electrode and the second electrode and where the controller is configured to receive one or more signals from the tissue adjacent to the distal end of the cannula and detect a change in property of the tissue as the cannula is advanced, and where the controller is further configured to determine a position of the distal end of the cannula during advancement into the body. Other embodiments of this aspect include corresponding computer systems, apparatus, and computer programs recorded on one or more computer storage devices, each configured to perform the actions of the methods.

Implementations may include one or more of the following features: The access device apparatus where the second electrode is positioned at or near the distal end of the cannula and the second lead runs through the inner lumen of the cannula. The apparatus where the second electrode is positioned on a skin of the body. The apparatus where the cannula includes a blunt distal tip. The apparatus where the first lead is embedded in a wall of the cannula. The apparatus where the first electrode is incorporated into a stylus that is configured to be inserted into the inner lumen of the cannula. The apparatus where the second electrode is incorporated into the stylus. The apparatus where the stylus is removable from the cannula. The apparatus where the stylus includes a stylus inner lumen. The apparatus which includes a locking mechanism to lock the stylus in place within the cannula. The apparatus where the cannula is inserted into the body manually. The apparatus where the cannula is inserted into the body automatically. The apparatus where the first lead includes an insulated portion of a shaft of the cannula and the first electrode includes an uninsulated portion of the shaft of the cannula. Implementations of the described techniques may include hardware, a method or process, or computer software on a computer-accessible medium.

One general aspect includes a method for accessing a portion of a body including advancing a cannula into the body so that the cannula contacts tissue adjacent to the cannula during advancement via a first electrode positioned at or near a distal end of the cannula. The method may also include positioning a second electrode so that it is in contact with the body. The method may also include receiving one or more signals from the tissue in contact with the first and second electrodes via the first lead and a second lead. The method may also include detecting a change in an impedance or a conductance of the tissue between the first electrode and the second electrode via a controller in communication with the first and second electrodes as the cannula is advanced. The method may also include determining, via the controller, a position of the cannula within the body based upon the change in the impedance or the conductance of the tissue between the first electrode and the second electrode as the cannula is advanced. The method may also include where the first lead runs through an inner lumen of the cannula. Other embodiments of this aspect include corresponding computer systems, apparatus, and computer programs recorded on one or more computer storage devices, each configured to perform the actions of the methods.

Implementations may include one or more of the following features: The method where the second electrode is positioned at or near the distal end of the cannula. The method where the second lead runs through the inner lumen of the cannula. The method where the cannula includes a blunt distal tip. The method where the first electrode is incorporated into a stylus that is configured to be inserted into the inner lumen of the cannula. The method where the second electrode is incorporated into the stylus. The method which includes a locking mechanism to lock the stylus in place within the cannula. The method where the first lead includes an insulated portion of a shaft of the cannula and the first electrode includes an uninsulated portion of the shaft of the cannula. Implementations of the described techniques may include hardware, a method or process, or computer software on a computer-accessible medium.

### BRIEF DESCRIPTION OF THE DRAWINGS

FIG. 1 shows an access device in the prior art.

FIG. 2 shows an embodiment of the conductive access device where the electrodes are incorporated into the threads of the access device.

FIG. 3 shows an embodiment of the conductive access device where at least one electrode is incorporated into the shaft of the device.

FIG. 4 shows an embodiment of the conductive access device where one of the electrodes is located at the distal tip of the access device.

FIG. 5 shows an embodiment of the conductive access device where at least one electrode is located on the distal edge of the access device.

FIG. 6 shows an embodiment of the conductive access device where the electrodes are incorporated into different threads of the access device.

FIG. 7 shows the distal tip of an embodiment of the conductive access device where 2 electrodes are at the blunt tip of the cannula.

FIG. 8 shows data collected in an in vivo porcine model to obtain conductivity measurements in real-time using an embodiment of the conductive access device.

FIG. 9A-B show an embodiment of the access device which includes a stylet.

FIG. 10A-B show an embodiment of the access device which incorporates a spring loaded or hydraulic stylet

FIG. 11 shows an embodiment of a stylet which is hollow.

FIG. 12 shows an embodiment of the access device with one electrode on the distal end of the stylet and one electrode on the distal end of the cannula component.

FIG. 13 shows a cross section of the access device with a solid stylet. Stylet

FIG. 14 shows a cross section of the access device with a hollow stylet.

FIG. 15 shows a cross section of the access device with and insulated lead within the inner lumen of the cannula component.

FIG. 16 shows a cross section of the access device with and insulated lead embedded within an insulated coating of the inner lumen of the cannula component.

FIG. 17 shows a cross section of the access device with two insulated leads within the inner lumen of the cannula component.

FIG. 18 shows a cross section of the access device with a lead embedded in an insulated coating of the inner lumen of the cannula.

FIG. 19 shows a cross section of the access device with two insulated leads within the inner lumen of the cannula component.

FIG. 20 shows the distal tip of an embodiment of the access device.

FIG. 21 shows the distal tip of an embodiment of the access device.

FIG. 22 shows the distal tip of an embodiment of the access device.

FIG. 23 shows the distal tip of an embodiment of the access device.

FIG. 25 shows an example of a data processing system.

### DETAILED DESCRIPTION

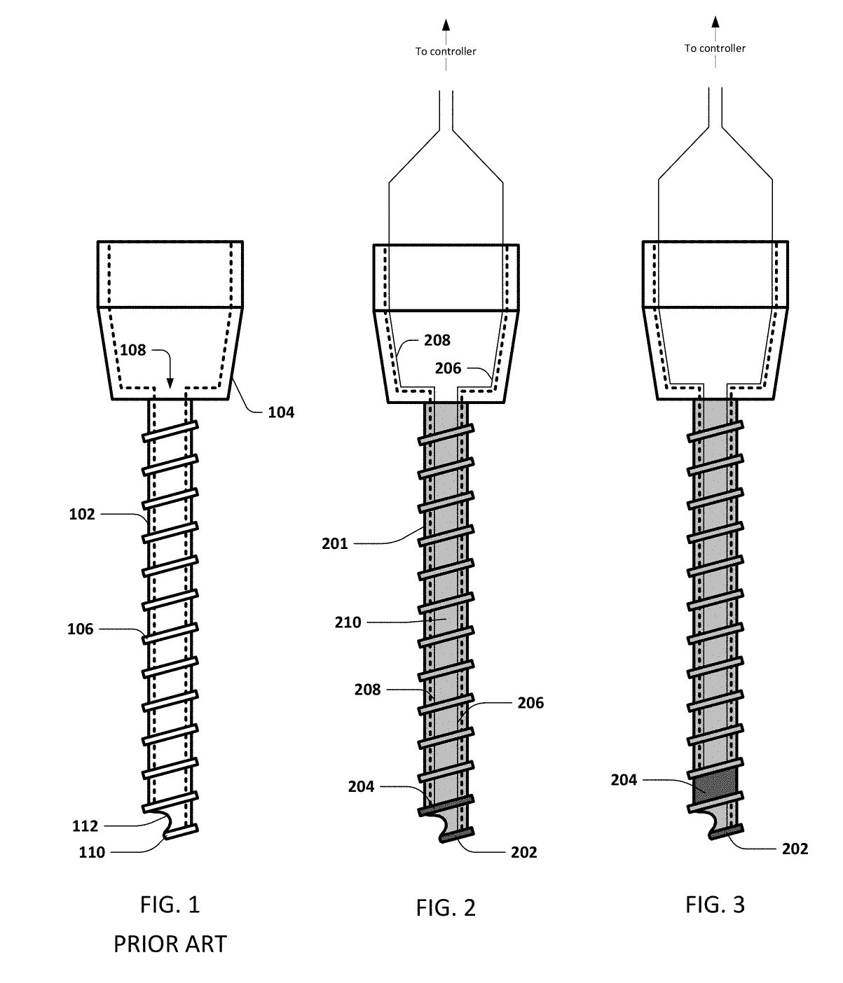

Access to body cavities or body tissue is often gained using a trocar, cannula, needle, catheter etc. These devices may be solid or hollow, sharp or blunt. For example, FIG. 1 shows a blunt hollow cannula, similar to that described in U.S. Pat. No. 5,478,329, which is hereby incorporated by reference in its entirety. The cannula shown in FIG. 1 includes shaft 102, head 104, threads 106, inner lumen 108, blunt tip 110, and optional concave cutting edge 112. This device is currently used to access body cavities/tissue by first making a small incision, then rotating the device into the body tissue. The threads help force the blunt tip through tissue as the cannula is rotated. The blunt tip helps to separate tissue, rather than cut tissue, which helps avoid severing blood vessels and causing major damage to tissue.

FIG. 2 shows an embodiment of a new conductive access device. The conductive access device may include a cannula with an inner lumen, threads and a blunt tip, or the conductive access device may be sharp, solid and/or non-threaded. FIG. 2 includes cannula 201, first electrode, or conductive surface, 202 and second electrode, or conductive surface, 204. The conductive access device may also include electrode leads 206 and 208, or one or more than one lead may be incorporated into the conductive access device shaft and/or head. For example, the leads may run down the inner wall of the device, as shown here, or one or more leads may be incorporated into the threads, the shaft wall etc. The leads may be insulated by a non-conductive coating, for example a polymer coating. An electrode may be simply an extension of a lead where the lead is insulated and the electrode is not insulated, for example, a wire, stylet, ribbon, cannula which is partially coated with an insulating material (the lead portion) and partially non-insulated (the electrode portion). An inner tube or coating, for example an extruded polymer tube may be added to the inner lumen of the device to protect the leads from damage from any tools that may be used through the cannula. The coating may alternatively/additionally for the lead portion of an electrode where the cannula comprises the lead and the electrode. The outer shaft of the conductive access device may include non-conductive coating 210 on all or part of its surface, as shown by the grey areas here. The conductive surfaces are shown as darker here, near the non-head, or distal, end of the device. In this embodiment, the shaft of the conductive access device may be made from a conductive material, such as metal. Most of the shaft of the conductive access device may be coated with a non-conductive coating such as a polymer. The coating may be shrink wrapped, sprayed on, dipped etc. Electrodes 202 and 204 are either masked, or left uncoated by the nonconductive coating. The coating may be on the outside of the shaft, the inside of the shaft, or both.

Alternatively, the conductive access device may be made from a non-conductive material, such as a hard polymer, and the electrodes may be made of a conductive material, such as metal. The conductive access device may be disposable or reusable. If it is made primarily from a polymer, it may be disposable, while a controller component may be reusable. If the conductive access device is made primarily from a metal, it may be reusable.

Leads 206 and 208 may pass through the head of the conductive access device, or may exit the device at any point. The leads are connected, either via wires, or wirelessly, to a controller which collects and may store the conductivity/impedance data from the electrodes, analyzes the data, and displays, or otherwise reports to the user the location of the conductive access device based on the conductivity/impedance data and/or changes in the conductivity/impedance data as the access device is advanced or moved through tissue. In some embodiments, one or more electrodes are placed at or near the distal tip of the conductive access device so that the location information informs the user of the location of the distal tip of the conductive access device.

In some embodiments, the controller may control the automatic application of force to the access device in either or both the advancing (into the body) direction and a rotational direction.

In some embodiments, the controller may have the capability to receive and analyze information from other sensors on the device or in the system. For example, the access device may incorporate pressure or force sensors, to avoid excessive force or to sense when force necessary for advancement is diminished, possibly indicating a body cavity has likely been accessed.

In use, some embodiments of the conductive access device are used as follows. As the device is advanced through body tissue toward the desired location, conductivity and/or impedance (hereafter referred to as “conductivity”) data between 2 or more electrodes is measured, effectively measuring the conductivity of the body tissue at or near the distal tip of the conductive access device. Because the conductivity of tissue is different for different tissue in different locations in the body, information relating to the location of the distal tip of the conductive access device can be obtained, and used to monitor access progress.

For example, the table below lists the electrical conductivity of various tissues up to 1 MHz. Other frequencies may be used by various embodiments of the conductive access device disclosed herein. The difference in conductivity can be used to identify the location of the conductive access device as it advances through tissue, and into different types of tissue.

| | | Standard | Number of | | |
|---|---|---|---|---|---|
| Conductivity (S/m) | Average | Deviation | Studies | Minimum | Maximum |
| Adrenal Gland | 4.81E−1 | 0.00E+0 | 1 | 4.81E−1 | 4.81E−1 |
| Air | 0.00E+0 | 0.00E+0 | 1 | 0.00E+0 | 0.00E+0 |
| Bile | 1.47E+0 | 2.80E−1 | 2 | 1.27E+0 | 1.67E+0 |
| Blood | 6.60E−1 | 1.39E−1 | 19 | 4.33E−1 | 9.46E−1 |
| Blood Plasma | 1.09E+0 | 8.49E−1 | 3 | 1.11E−1 | 1.59E+0 |
| Blood Serum | 1.18E+0 | 4.38E−1 | 3 | 7.25E−1 | 1.60E+0 |
| Blood Vessel Wall | 2.32E−1 | 0.00E+0 | 1 | 2.32E−1 | 2.32E−1 |
| Bone (Cancellous) | 8.21E−2 | 7.82E−2 | 3 | 3.60E−2 | 1.72E−1 |
| Bone (Cortical) | 3.50E−3 | 2.11E−3 | 3 | 1.85E−3 | 5.88E−3 |
| Bone (Cortical)-Across | 1.85E−3 | 0.00E+0 | 1 | 1.85E−3 | 1.85E−3 |
| Bone (Cortical)-Along | 5.88E−3 | 0.00E+0 | 1 | 5.88E−3 | 5.88E−3 |
| Bone (Cortical)-Around | 2.78E−3 | 0.00E+0 | 1 | 2.78E−3 | 2.78E−3 |
| Bone Marrow (Red) | 0.00E+0 | 0.00E+0 | 0 | 0.00E+0 | 0.00E+0 |
| Bone Marrow (Yellow) | 2.47E−3 | 0.00E+0 | 1 | 2.47E−3 | 2.47E−3 |
| Brain | 2.34E−1 | 1.50E−1 | 4 | 1.10E−1 | 4.51E−1 |
| Brain (Grey Matter) | 2.39E−1 | 1.13E−1 | 8 | 1.09E−1 | 4.81E−1 |
| Brain (Grey Matter) x-along | 2.55E−1 | 1.06E−1 | 2 | 1.80E−1 | 3.30E−1 |
| Brain (Grey Matter) y-across | 1.95E−1 | 3.54E−2 | 2 | 1.70E−1 | 2.20E−1 |
| Brain (Grey Matter) z-normal to | 2.15E−1 | 9.19E−2 | 2 | 1.50E−1 | 2.80E−1 |
| surface axis | | | | | |
| Brain (White Matter) | 2.65E−1 | 3.83E−1 | 8 | 6.44E−2 | 1.20E+0 |
| Brain (White Matter) across fibers | 1.28E−1 | 6.88E−3 | 4 | 1.18E−1 | 1.33E−1 |
| Brain (White Matter) longitudial | 1.20E+0 | 9.15E−2 | 4 | 1.12E+0 | 1.28E+0 |
| (parallel to fibers) | | | | | |
| Breast Fat | 2.22E−2 | 0.00E+0 | 1 | 2.22E−2 | 2.22E−2 |
| Breast Gland | 0.00E+0 | 0.00E+0 | 0 | 0.00E+0 | 0.00E+0 |
| Bronchi | 2.32E−1 | 0.00E+0 | 1 | 2.32E−1 | 2.32E−1 |
| Bronchi lumen | 0.00E+0 | 0.00E+0 | 1 | 0.00E+0 | 0.00E+0 |
| Cartilage | 1.01E+0 | 0.00E+0 | 1 | 1.01E+0 | 1.01E+0 |
| Cerebellum | 6.60E−1 | 4.43E−1 | 6 | 2.20E−1 | 1.31E+0 |
| Cerebellum x | 1.21E+0 | 1.36E−1 | 2 | 1.12E+0 | 1.31E+0 |
| Cerebellum y | 3.10E−1 | 1.27E−1 | 2 | 2.20E−1 | 4.00E−1 |
| Cerebellum z | 4.55E−1 | 4.95E−2 | 2 | 4.20E−1 | 4.90E−1 |
| Cerebrospinal Fluid | 1.78E+0 | 5.89E−2 | 12 | 1.59E+0 | 1.80E+0 |
| Cervix | 3.38E−1 | 0.00E+0 | 1 | 3.38E−1 | 3.38E−1 |
| Commissura Anterior | 2.65E−1 | 3.83E−1 | 8 | 6.44E−2 | 1.20E+0 |
| Commissura Anterior-Across | 1.28E−1 | 6.88E−3 | 4 | 1.18E−1 | 1.33E−1 |
| Commissura Posterior | 2.65E−1 | 3.83E−1 | 8 | 6.44E−2 | 1.20E+0 |
| Commissura Posterior-Across | 1.28E−1 | 6.88E−3 | 4 | 1.18E−1 | 1.33E−1 |
| Commissura Posterior-Along | 1.20E+0 | 9.15E−2 | 4 | 1.12E+0 | 1.28E+0 |
| Commissura Posterior-Along | 1.20E+0 | 9.15E−2 | 4 | 1.12E+0 | 1.28E+0 |
| Connective Tissue | 0.00E+0 | 0.00E+0 | 0 | 0.00E+0 | 0.00E+0 |
| Diaphragm | 3.55E−1 | 1.99E−1 | 18 | 2.00E−2 | 6.70E−1 |
| Diaphragm-Parallel | 4.35E−1 | 1.89E−1 | 5 | 1.50E−1 | 6.67E−1 |
| Diaphragm-Perpendicular | 1.75E−1 | 1.47E−1 | 5 | 4.35E−2 | 4.17E−1 |
| Dorsal Column | 2.77E−1 | 2.75E−1 | 2 | 8.26E−2 | 4.72E−1 |
| Dorsal Column-Across | 8.26E−2 | 0.00E+0 | 1 | 8.26E−2 | 8.26E−2 |
| Dorsal Column-Along | 4.72E−1 | 0.00E+0 | 1 | 4.72E−1 | 4.72E−1 |
| Ductus Deferens | 2.32E−1 | 0.00E+0 | 1 | 2.32E−1 | 2.32E−1 |
| Dura | 3.68E−1 | 0.00E+0 | 1 | 3.68E−1 | 3.68E−1 |
| Epididymis | 3.66E−1 | 0.00E+0 | 1 | 3.66E−1 | 3.66E−1 |
| Esophagus | 1.64E−1 | 0.00E+0 | 1 | 1.64E−1 | 1.64E−1 |
| Esophagus Lumen | 0.00E+0 | 0.00E+0 | 1 | 0.00E+0 | 0.00E+0 |
| Eye (Cornea) | 6.20E−1 | 0.00E+0 | 1 | 6.20E−1 | 6.20E−1 |
| Eye (Lens) | 3.45E−1 | 0.00E+0 | 1 | 3.45E−1 | 3.45E−1 |
| Eye (Sclera) | 6.20E−1 | 0.00E+0 | 1 | 6.20E−1 | 6.20E−1 |
| Eye (Vitreous Humor) | 1.55E+0 | 0.00E+0 | 1 | 1.55E+0 | 1.55E+0 |
| Eye Lens (Cortex) | 3.40E−1 | 0.00E+0 | 1 | 3.40E−1 | 3.40E−1 |
| Eye Lens (Nucleus) | 1.90E−1 | 0.00E+0 | 1 | 1.90E−1 | 1.90E−1 |
| Fat | 5.73E−2 | 4.87E−2 | 8 | 2.40E−2 | 1.70E−1 |
| Fat (Average Infiltrated) | 5.73E−2 | 4.87E−2 | 8 | 2.40E−2 | 1.70E−1 |
| Fat (Not Infiltrated) | 5.73E−2 | 4.87E−2 | 8 | 2.40E−2 | 1.70E−1 |
| Gallbladder | 4.08E−1 | 4.28E−2 | 7 | 3.51E−1 | 4.76E−1 |
| Heart Lumen | 6.60E−1 | 1.39E−1 | 19 | 4.33E−1 | 9.46E−1 |
| Heart Muscle | 3.81E−1 | 1.75E−1 | 28 | 1.04E−1 | 6.46E−1 |
| Heart Muscle Longitudial | 3.94E−1 | 7.62E−2 | 3 | 3.19E−1 | 4.72E−1 |
| Heart Muscle Transversal | 2.16E−1 | 4.47E−2 | 3 | 1.77E−1 | 2.65E−1 |
| Hippocampus | 2.76E−1 | 1.38E−1 | 5 | 1.30E−1 | 4.81E−1 |
| Hypophysis | 4.81E−1 | 0.00E+0 | 1 | 4.81E−1 | 4.81E−1 |
| Hypothalamus | 2.39E−1 | 1.13E−1 | 8 | 1.09E−1 | 4.81E−1 |
| Intervertebral Disc | 1.01E+0 | 0.00E+0 | 1 | 1.01E+0 | 1.01E+0 |
| Kidney | 4.03E−1 | 2.89E−1 | 7 | 1.42E−1 | 9.01E−1 |
| Kidney (Cortex) | 4.46E−1 | 2.91E−1 | 6 | 1.67E−1 | 9.01E−1 |
| Kidney (Medulla) | 4.46E−1 | 2.91E−1 | 6 | 1.67E−1 | 9.01E−1 |
| Large Intestine | 1.64E−1 | 0.00E+0 | 1 | 1.64E−1 | 1.64E−1 |
| Large Intestine Lumen | 3.55E−1 | 1.99E−1 | 18 | 2.00E−2 | 6.70E−1 |
| Larynx | 1.01E+0 | 0.00E+0 | 1 | 1.01E+0 | 1.01E+0 |
| Liver | 2.21E−1 | 3.52E−1 | 27 | 6.37E−2 | 1.95E+0 |
| Lung | 1.05E−1 | 5.48E−2 | 10 | 5.81E−2 | 2.49E−1 |
| Lung (Deflated) | 5.29E−1 | 1.70E−1 | 27 | 4.18E−2 | 8.05E−1 |
| Lung (Inflated) | 4.64E−1 | 1.81E−1 | 28 | 4.20E−2 | 7.37E−1 |
| Lymph | 0.00E+0 | 0.00E+0 | 0 | 0.00E+0 | 0.00E+0 |
| Lymphnode | 2.93E−1 | 1.66E−1 | 8 | 1.07E−1 | 4.80E−1 |
| Mandible | 3.50E−3 | 2.11E−3 | 3 | 1.85E−3 | 5.88E−3 |
| Medulla Oblongata | 2.34E−1 | 1.50E−1 | 4 | 1.10E−1 | 4.51E−1 |
| Meniscus | 1.01E+0 | 0.00E+0 | 1 | 1.01E+0 | 1.01E+0 |
| Midbrain | 2.34E−1 | 1.50E−1 | 4 | 1.10E−1 | 4.51E−1 |
| Mucous Membrane | 3.55E−1 | 1.99E−1 | 18 | 2.00E−2 | 6.70E−1 |
| Muscle | 3.55E−1 | 1.99E−1 | 18 | 2.00E−2 | 6.70E−1 |
| Muscle Parallel | 4.35E−1 | 1.89E−1 | 5 | 1.50E−1 | 6.67E−1 |
| Muscle Perpendicular | 1.75E−1 | 1.47E−1 | 5 | 4.35E−2 | 4.17E−1 |
| Nerve | 2.65E−1 | 3.83E−1 | 8 | 6.44E−2 | 1.20E+0 |
| Nerve-Across | 1.28E−1 | 6.88E−3 | 4 | 1.18E−1 | 1.33E−1 |
| Nerve-Along | 1.20E+0 | 9.15E−2 | 4 | 1.12E+0 | 1.28E+0 |
| Ovary | 3.66E−1 | 0.00E+0 | 1 | 3.66E−1 | 3.66E−1 |
| Pancreas | 1.45E−1 | 2.13E−2 | 2 | 1.30E−1 | 1.60E−1 |
| Penis | 2.32E−1 | 0.00E+0 | 1 | 2.32E−1 | 2.32E−1 |
| Pharynx | 0.00E+0 | 0.00E+0 | 1 | 0.00E+0 | 0.00E+0 |
| Pineal Body | 4.81E−1 | 0.00E+0 | 1 | 4.81E−1 | 4.81E−1 |
| Placenta | 6.60E−1 | 1.39E−1 | 19 | 4.33E−1 | 9.46E−1 |
| Pons | 2.34E−1 | 1.50E−1 | 4 | 1.10E−1 | 4.51E−1 |
| Prostate | 6.70E−1 | 9.00E−2 | 3 | 5.70E−1 | 7.43E−1 |
| SAT (Subcutaneous Fat) | 5.73E−2 | 4.87E−2 | 8 | 2.40E−2 | 1.70E−1 |
| Salivary Gland | 6.70E−1 | 9.00E−2 | 3 | 5.70E−1 | 7.43E−1 |
| Seminal Vesicle | 6.70E−1 | 9.00E−2 | 3 | 5.70E−1 | 7.43E−1 |
| Skin | 1.70E−1 | 1.13E−1 | 2 | 9.00E−2 | 2.50E−1 |
| Skull (Cancellous) | 3.20E−1 | 0.00E+0 | 1 | 3.20E−1 | 3.20E−1 |
| Skull (Cortical) | 3.20E−1 | 0.00E+0 | 1 | 3.20E−1 | 3.20E−1 |
| Small Intestine | 1.64E−1 | 0.00E+0 | 1 | 1.64E−1 | 1.64E−1 |
| Small Intestine Lumen | 3.55E−1 | 1.99E−1 | 18 | 2.00E−2 | 6.70E−1 |
| Spinal Cord | 2.34E−1 | 1.50E−1 | 4 | 1.10E−1 | 4.51E−1 |
| Spleen | 2.93E−1 | 1.66E−1 | 8 | 1.07E−1 | 4.80E−1 |
| Stomach | 1.64E−1 | 0.00E+0 | 1 | 1.64E−1 | 1.64E−1 |
| Stomach Lumen | 3.55E−1 | 1.99E−1 | 18 | 2.00E−2 | 6.70E−1 |
| Tendon\Ligament | 3.68E−1 | 0.00E+0 | 1 | 3.68E−1 | 3.68E−1 |
| Testis | 3.66E−1 | 0.00E+0 | 1 | 3.66E−1 | 3.66E−1 |
| Thalamus | 2.39E−1 | 1.13E−1 | 8 | 1.09E−1 | 4.81E−1 |
| Thymus | 1.75E−1 | 0.00E+0 | 2 | 5.73E−2 | 2.93E−1 |
| Thyroid Gland | 4.81E−1 | 0.00E+0 | 1 | 4.81E−1 | 4.81E−1 |
| Tongue | 3.55E−1 | 1.99E−1 | 18 | 2.00E−2 | 6.70E−1 |
| Tooth | 3.50E−3 | 2.11E−3 | 3 | 1.85E−3 | 5.88E−3 |
| Tooth (Dentine) | 3.50E−3 | 2.11E−3 | 3 | 1.85E−3 | 5.88E−3 |
| Tooth (Enamel) | 3.50E−3 | 2.11E−3 | 3 | 1.85E−3 | 5.88E−3 |
| Trachea | 3.42E−1 | 0.00E+0 | 1 | 3.42E−1 | 3.42E−1 |
| Trachea Lumen | 0.00E+0 | 0.00E+0 | 1 | 0.00E+0 | 0.00E+0 |
| Ureter\Urethra | 2.32E−1 | 0.00E+0 | 1 | 2.32E−1 | 2.32E−1 |
| Urinary Bladder Wall | 4.08E−1 | 4.28E−2 | 7 | 3.51E−1 | 4.76E−1 |
| Urine | 2.95E+0 | 5.44E−1 | 2 | 2.56E+0 | 3.33E+0 |
| Uterus | 3.91E−1 | 0.00E+0 | 1 | 3.91E−1 | 3.91E−1 |
| Vagina | 1.64E−1 | 0.00E+0 | 1 | 1.64E−1 | 1.64E−1 |
| Vertebrae | 3.50E−3 | 2.11E−3 | 3 | 1.85E−3 | 5.88E−3 |
| Water | 0.00E+0 | 0.00E+0 | 0 | 0.00E+0 | 0.00E+0 |

### From the IT′IS Foundation

In some embodiments, the conductive access device may be rotated/advanced automatically via a motor connected to the controller. The motor may automatically start, stop, slow or reverse based on signals from the controller based on data received from the device. For example, the motor may stop when the conductivity across the electrodes of the device indicate that the distal tip of the device is in the abdominal cavity, if this is the desired final location of the device. Alternatively, the motor may slow or stop if the amount of force required to advance the device is too high.

FIG. 3 shows an embodiment of the conductive access device where at least one of the electrodes is incorporated into the shaft surface of the cannula. In this figure, electrode 202 is incorporated into a thread while electrode 204 is incorporated into a surface of the shaft. This may be done by leaving part of the shaft uncoated with insulating material, similar to the examples mentioned in association with FIG. 2. The leads to the electrodes may run down the inner lumen of the device, under the insulating coating, within the insulating coating, within a spiraling thread or elsewhere.

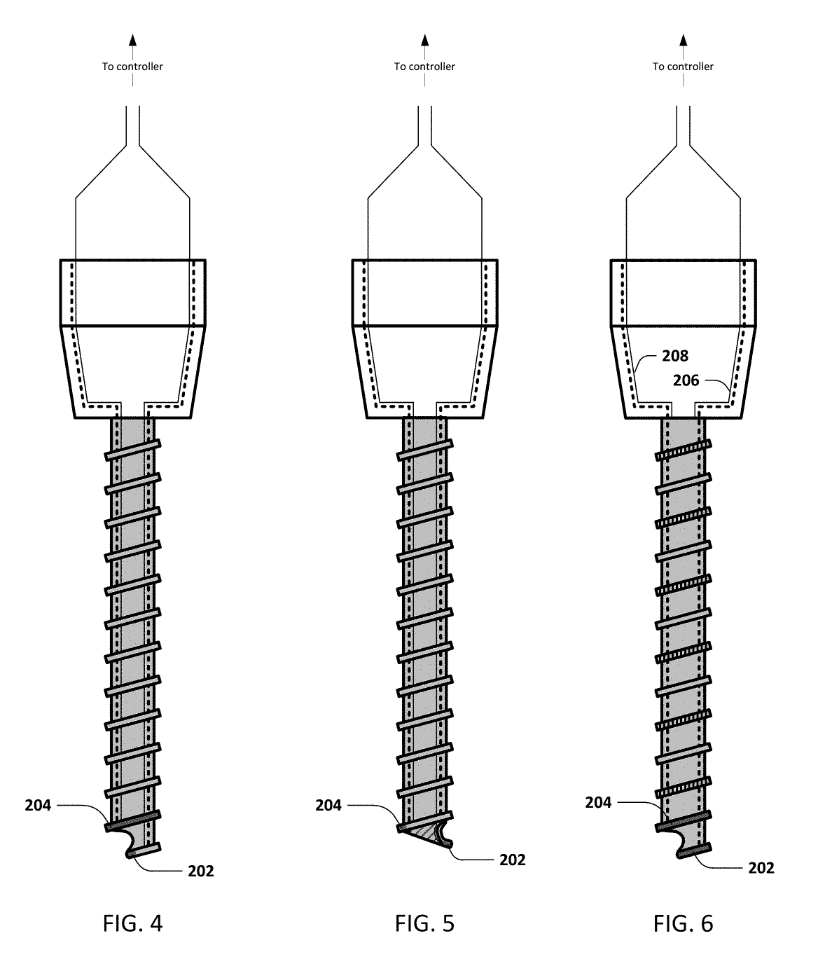

FIG. 4 shows an embodiment of the conductive access device where at least one electrode is at the most distal tip of the cannula. This Figure shows an example where the most distal electrode is at the most distal blunt tip of the cannula.

FIG. 5 shows an embodiment of the conductive access device where at least one electrode is on the distal edge and/or inside of the lumen of the cannula. At least one electrode may also be completely inside the lumen of the cannula. This placement may reduce the chances of damage to the electrode as the device is being used/advanced. An inner protective sheath may be used to protect all or a portion of the leads and/or electrodes. Alternatively or additionally, channels or guides may be used within the cannula shaft and/or lumen to protect and/or guide the leads.

FIG. 6 shows an embodiment of the conductive access device where 2 electrodes are incorporated into separate thread lines. In this embodiment, 2 or more separate sets of threads are used on the outside of the shaft. In this embodiment, the leads for the electrodes may be incorporated into the threads so that each lead has its own set of threads and the 2 sets of threads are not in contact with each other. In this (and other) embodiment, leads 206 and 208 may be embedded into the threads themselves. The leads may even be the threads, if they are properly insulated for most of the length of the shaft, revealing the electrodes at the distal end where there is no insulation. Alternatively, or additionally, the cannula, itself, may serve as a lead, by insulating at least a portion of the cannula.

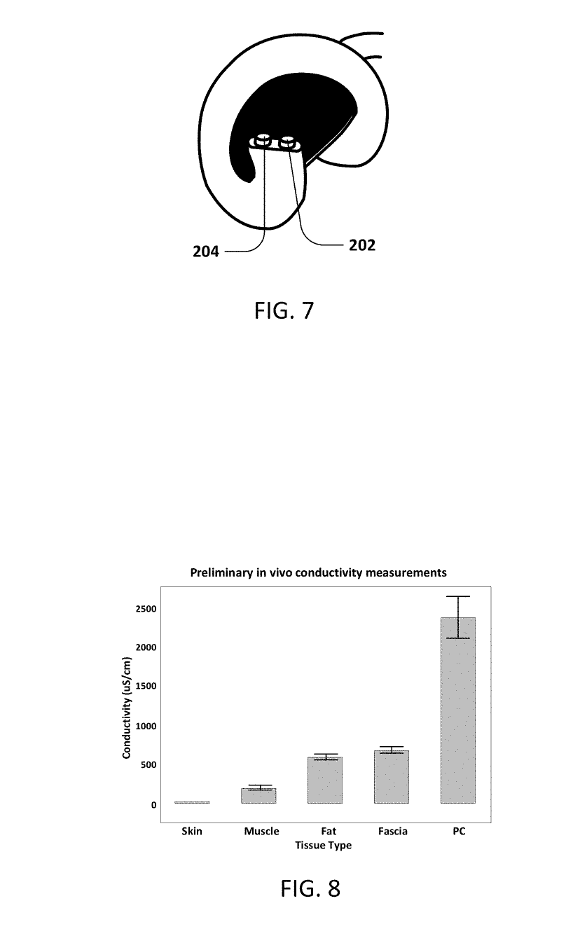

FIG. 7 shows the distal tip of an embodiment of the conductive access device where 2 electrodes are at the blunt tip of the cannula. As with any of the embodiments disclosed herein, more electrodes may be present, for example, 4 electrodes. As with any of the embodiments disclosed herein, the access device may have a blunt tip, or may have a sharp tip.

FIG. 8 shows data collected in an in vivo porcine model to obtain conductivity measurements in real-time using an embodiment of the conductive access device. A midline incision was made at the level of the umbilicus to allow for visual confirmation of anatomical placement of the device. Measurements were taken at the level of the skin, the subcutaneous fat, fascia, muscle, and peritoneal cavity. The cannula was inserted to each tissue layer ten times, and placement was confirmed visually and with fluoroscopy. A separate conductivity measurement was taken upon each insertion resulting in a total of 50 measurements total (10 per tissue type). Data were analyzed using a repeated measures ANOVA to determine differences in conductivity measurements between tissue types, the results of which are shown in FIG. 8. The results of the post-hoc Tukey multiple comparisons reveal a significant difference between the layers of the abdominal wall including the skin and the subcutaneous fat (p=0.0083), the skin and the muscle (p<0.0001), the skin and fascia (p=0.0027), and the subcutaneous fat and the muscle (p=0.01251). In addition, the results reveal a significantly higher conductivity (p<0.0001) for the peritoneal cavity by an order of magnitude when compared to each of the layers of the abdominal wall. These data show that electrical conductivity can be used to determine positioning of the conductive access device in real time as it passes through the layers of the abdominal wall and into the peritoneal cavity.

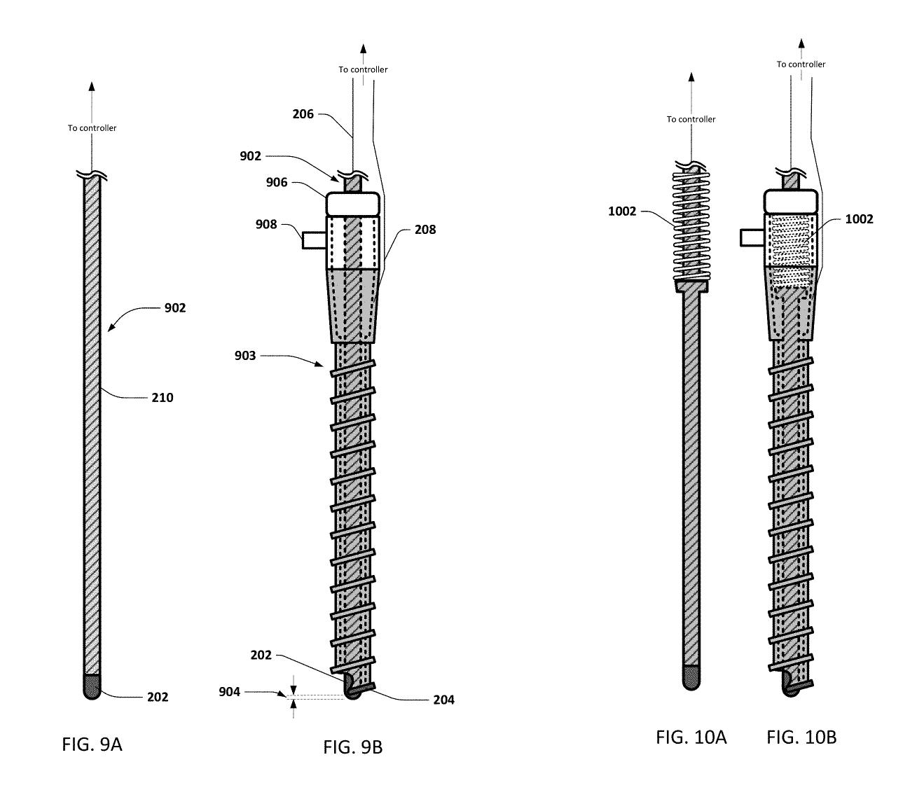

FIG. 9A-B show an embodiment of the access device which includes a stylet. Stylet 902 is configured to fit through the lumen of cannula component 903. Stylet 902 includes electrode portion 202 and may be made out of polymer, metal, or other suitable material. If the stylet is made out of metal, insulating coating 210 may be incorporated to insulate the bulk of the stylet. The stylet is electrically connected to the controller via wires or other means. Stylet 902 may be solid or hollow, with a lumen down its length. Stylet may be blunt tipped, sharp tipped, flat tipped, or shaped in any way at the tip.

FIG. 9B shows the stylet inside the lumen of the cannula component. In this figure, the stylet is solid, without an inner lumen. The distal tip of the stylet serves as one electrode 202 and the distal tip of the cannula component serves as the other electrode 204. In this embodiment, both the stylet and the cannula component electrodes are electrically connected to the controller via leads 206 and 208. In this embodiment, the distal tip of stylet 902 is configured to be fixed distance 904 from the distal end of the cannula component. The fixed distance may be positive, so that the stylet protrudes from the cannula component, negative, so the stylet is recessed into the cannula component, or zero, where the stylet is flush with the cannula component. In this embodiment, the inside lumen of the cannula may not need to be insulated.

To maintain distance 904 during use of the device, locking mechanism 906 may be incorporated into the cannula component to lock the position of the stylet with respect to the cannula component. Locking mechanism 906 may be loosened and relocked in the same or different positions as appropriate. Stylet 902 may be removable from the cannula by unlocking locking mechanism 906.

In embodiments where a solid stylet is used, fluid (gas or liquid, for example CO2) may be infused or removed via port 908 in the cannula component. In these embodiments, the stylet may be removed before the inner lumen of the cannula component is used for fluid/instruments, or, fluid may be infused/removed via the annular space between the inner lumen of the cannula component and the outer surface of the stylet. Alternatively, another lumen may be incorporated into the cannula component.

FIG. 10A-B show an embodiment of the access device which incorporates a spring loaded or hydraulic stylet. In this embodiment, spring (or other force capacitor) 1002 is incorporated into the assembly so that the spring is in compression when the tip of the access device is in solid tissue, and the spring extends when the tip of the access device is in open anatomy or a hollow organ, such as in the peritoneal cavity. The force exerted by the spring keeps the distal end of the stylet protruding beyond the distal end of the distal tip of the access device. This helps prevent inadvertent damage to internal organs, for example where the access device bumps up against more mobile tissue areas such as bowel, bladder etc. This operation is similar to that of a Veress needle. These embodiments may or may not include a locking mechanism. These embodiments, like all embodiments disclosed herein, may include a blunt or a sharp distal tip.

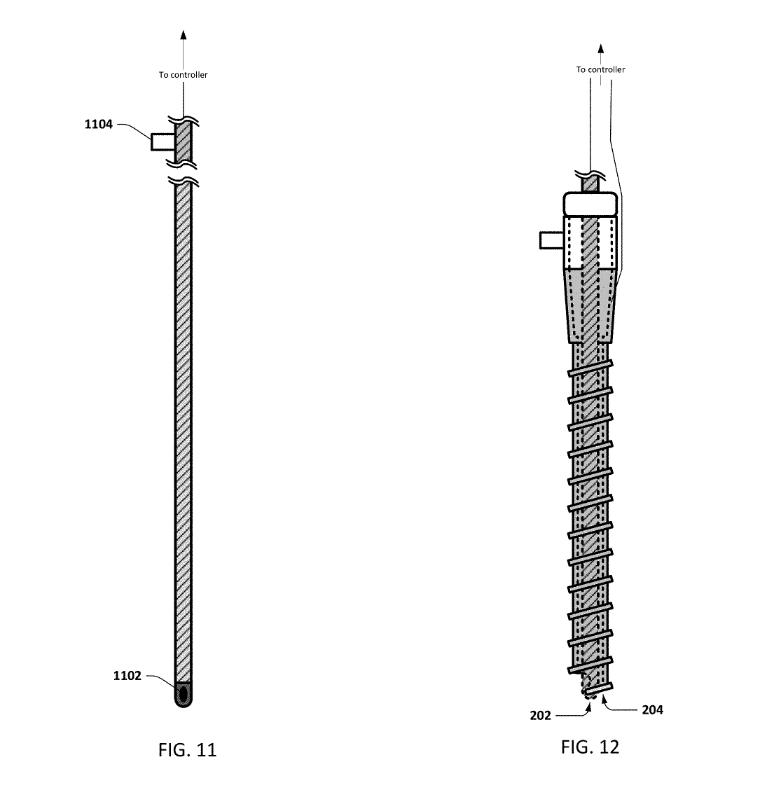

FIG. 11 shows an embodiment of a stylet which is hollow. The stylet has a lumen running down the length of it, from port 1104 to opening 1102. Fluid may be infused and/or removed through the stylet lumen while the stylet is in the access device.

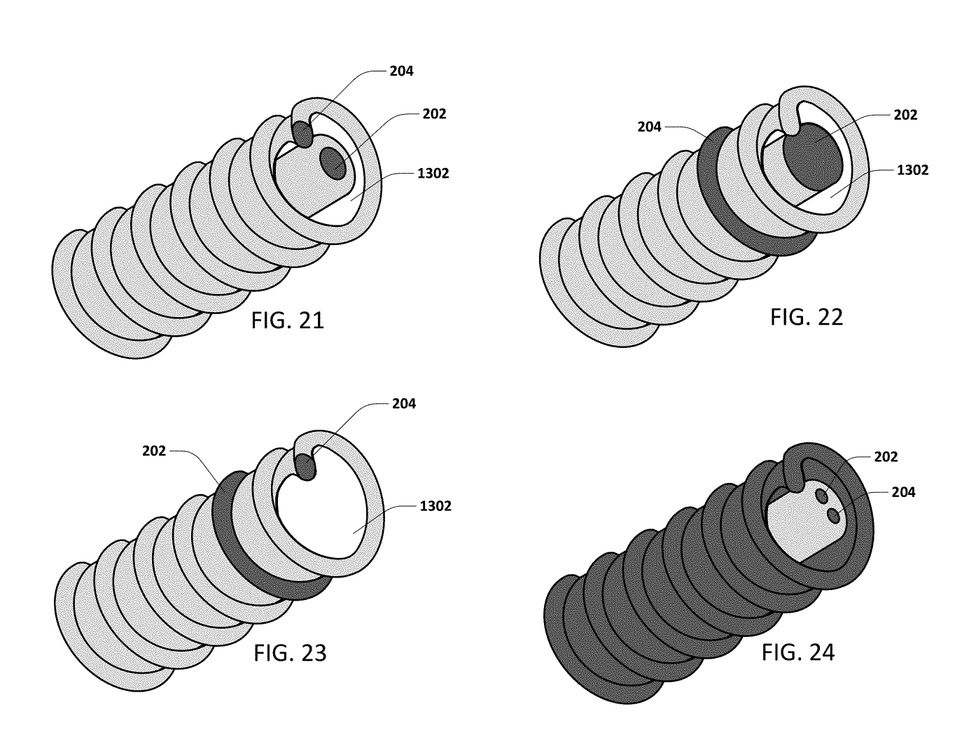

FIG. 12 shows an embodiment of the access device with one electrode, electrode 202, on the distal end of the stylet, and one electrode, electrode 204, on the distal end of the cannula component. The electrodes are not visible in this figure because electrode 202 is on the distal surface of the stylet. In other words, it is insulated so that electrode 202 does not contact the inner surface of the cannula component. Electrode 204 is on the inner surface of the inner lumen of the cannula component. This is also shown in FIG. 20. This configuration helps prevent the two electrodes from ever contacting each other accidentally.

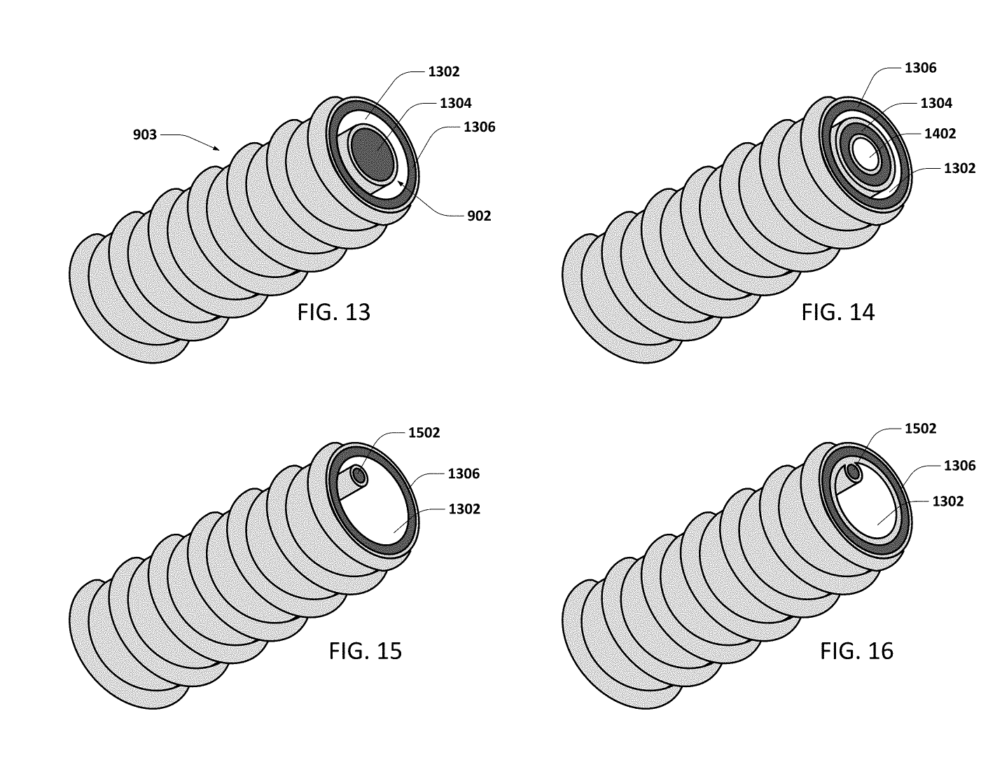

FIG. 13 shows a cross section of the access device with a solid stylet. Stylet 902 is shown with insulation on the majority of its length. Cannula component 903 is also shown with an insulating coating on the majority of its length. Metal core 1304 of stylet serves as a portion of the lead for one of the electrodes at the distal tip of the device. Metal core 1306 of the cannula component serves as a portion of the lead for another electrode at the distal tip of the device. In this embodiment, lumen 1302 (here shown as an annular lumen) may be used for infusion and/or removal of fluid. Alternatively, stylet 902 may be removed from cannula component 903 to access the inner lumen of the cannula.

FIG. 14 shows a cross section of the access device with a hollow stylet. In this embodiment, stylet inner lumen 1402 may be used for infusion and/or removal of fluids. Alternatively, or additionally, the annular lumen between the stylet and the cannula may be used, or the stylet may be removed from the cannula component.

FIG. 15 shows a cross section of the access device with and insulated lead within the inner lumen of the cannula component. Insulated lead 1502 serves as a portion of the lead for one of the electrodes at the distal tip of the device. Lumen 1302 may be used for infusion and/or removal of fluids or for instrument access.

FIG. 16 shows a cross section of the access device with and insulated lead embedded within an insulated coating of the inner lumen of the cannula component.

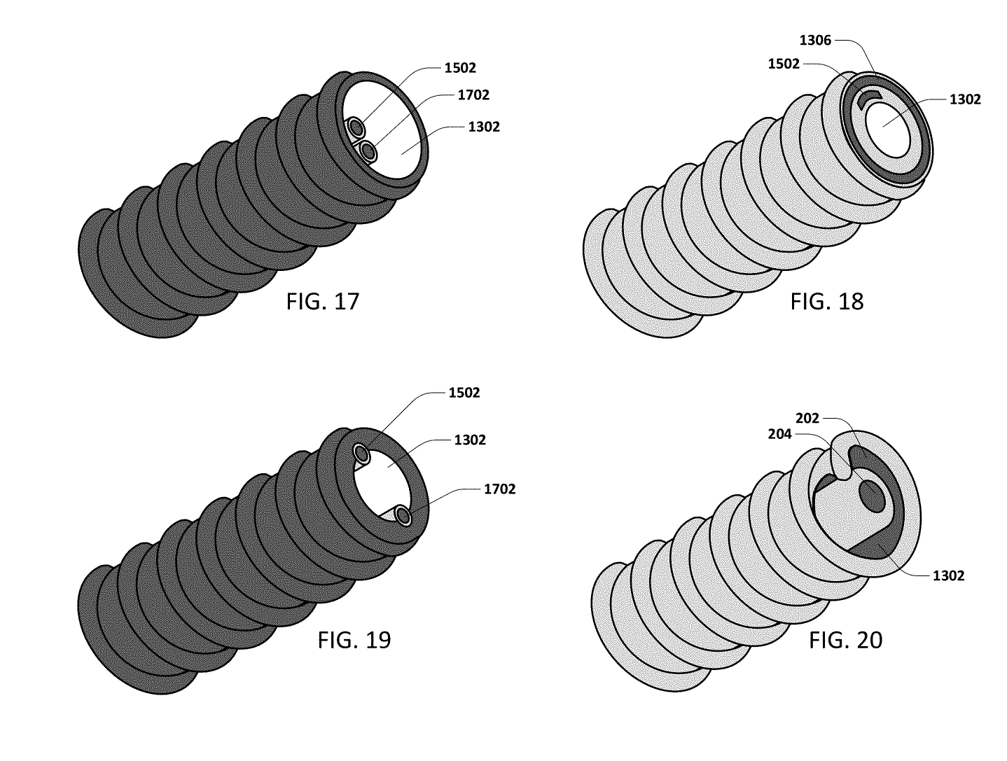

FIG. 17 shows a cross section of the access device with two insulated leads within the inner lumen of the cannula component. Insulated lead 1502 serves as a portion of the lead for one of the electrodes at the distal tip of the device. Insulated lead 1702 serves as a portion of the lead for another of the electrodes at the distal tip of the device. Lumen 1302 may be used for infusion and/or removal of fluids or for instrument access. Note that the cannula portion of this embodiment need not be coated with an insulated material. Coated leads 1502 and 1702 may be free-floating within lumen 1302, or may be attached to the wall of the cannula lumen along all or part of each of their lengths.

FIG. 18 shows a cross section of the access device with a lead embedded in an insulated coating of the inner lumen of the cannula. Lead 1502 may be flattened, i.e. a ribbon which is wider than it is thick, or any other appropriate shape.

FIG. 19 shows a cross section of the access device with two insulated leads within the inner lumen of the cannula component. In this embodiment, one or more coated leads is attached to the cannula inner surface via grooves or notches.

FIG. 20 shows the distal tip of an embodiment of the access device. In this embodiment, one electrode, electrode 202, is incorporated into the inner surface of the cannula, and one electrode is incorporated into the distal surface of the distal end of the stylet. An insulating coating covers most of the inner surface of the cannula, leaving the distal end serving as an electrode. Alternatively, the ID of the cannula may be uncoated, where the area between the OD of the stylet and the ID of the cannula is small enough, so that tissue may not enter between the two. The insulating coating of the stylet prevents electrode 204 from coming in contact with electrode 202 accidentally.

FIG. 21 shows the distal tip of an embodiment of the access device. In this embodiment, one electrode is incorporated into the distal end of the stylet, and the other is incorporated into the most distal tip of the cannula.

FIG. 22 shows the distal tip of an embodiment of the access device. In this embodiment, one electrode is incorporated into the distal end of the stylet, and the other is incorporated into a thread near the distal tip of the cannula.

FIG. 23 shows the distal tip of an embodiment of the access device. In this embodiment, one electrode is incorporated into the distal end of the cannula, and the other is incorporated into a thread near the distal tip of the cannula. In this embodiment, similar to that shown in FIG. 6, leads for the two electrodes may be incorporated into separate threads. Alternatively, the leads may run down the length of the cannula via any of the methods disclosed herein.

FIG. 24 shows the distal tip of an embodiment of the access device. In this embodiment, both electrodes are incorporated into the distal end of the stylet. In this embodiment, the cannula portion may be uncoated metal. The stylet may be an extrusion of polymer with one, two, or more electrodes and electrode leads embedded in the insulating polymer.

Some examples of procedures where the conductive access device may be useful include, laparoscopy, endoscopy, tissue sampling, biopsy, tracheotomy, vascular access, natural body lumen access (i.e. bowel, bladder, stomach, lung access), central nervous system access, lung access, amniotic access, tumor access, etc.

Some embodiments of the conductive access device may include a force sensor, pressure sensor, or other type of sensor at or near the distal tip of the device, or elsewhere. Some embodiments of the conductive access device controller may include a force detection component which monitors the amount of force necessary to automatically advance the access device, in either or both the rotational direction and the direction toward the inside of the patient's body.

Some embodiments of the access device may use automatic insertion of the device, controlled by the controller. Some embodiments of the access device may be manual, where the device is inserted manually into the patient's body.

Some embodiments may use only one electrode. Some embodiments may use 2 electrodes, 3 electrodes, 4 electrodes or more electrodes. Embodiments which use 1 electrode, may include a reference electrode patch or other type of electrode configured to be placed on the outside of the patient's body, or elsewhere on or in the patient's body.

Some embodiments may incorporate ablation and/or cauterizing ability into the access device. For example, the same electrodes used for measuring conductance/impedance may be used to cauterize or ablate tissue. The sensing and treatment functions may alternate. For example, conductance/impedance measurements may be used to determine the extent of cauterization and/or ablation of tissue. As tissue is cauterized and/or ablated, the hydration of the tissue decreases which in turn reduces the conductivity of the tissue. This conductivity can be monitored to determine the extent of cauterization/ablation. Alternatively, separate electrodes may be used for cauterization/ablation functions and location functions.

The inner diameter of the cannula portion of the access device may be around 1.6 mm. Alternatively, the inner diameter of the cannula portion of the access device may be around 1.2-2 mm. Alternatively, the inner diameter of the cannula portion of the access device may be around 2-5 mm. Alternatively, the inner diameter of the cannula portion of the access device may be around 5-10 mm.

The outer diameter of the stylet may be around 1.6 mm. Alternatively, the outer diameter of the stylet may be around 1.2-2 mm. Alternatively, the outer diameter of the stylet may be around 2-5 mm. Alternatively, the outer diameter of the stylet may be around 5-10 mm.

The outer diameter of the cannula portion of the access device may be around 2 mm. Alternatively, the outer diameter of the cannula portion of the access device may be around 1.5-2.5 mm. Alternatively, the outer diameter of the cannula portion of the access device may be around 2.5-5 mm. Alternatively, the outer diameter of the cannula portion of the access device may be around 5-10 mm.

Some embodiments may incorporate imaging capabilities, such as fiber optics through, or in conjunction with, the access device. The controller may process the images, or images may be viewed directly by the user or both. The controller may collect image processing data, such as the presence of adhesions, tumors, abnormalities etc., and this data may be used to correlate the presence or absence of certain features with conductance/impedance of tissue, force necessary for advancement through tissue, etc. This correlation may then be used to help guide the access device.

Example of Data Processing System

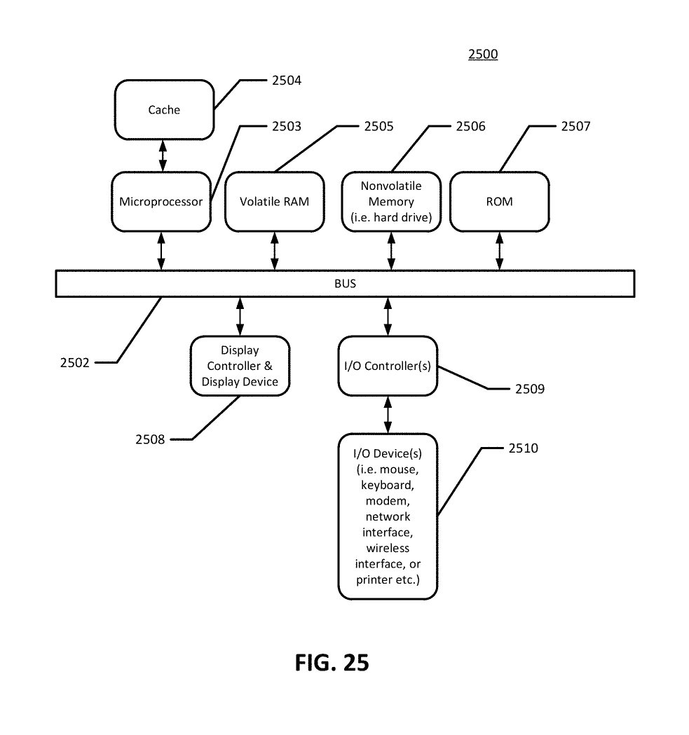

FIG. 25 is a block diagram of a data processing system, which may be used with any embodiment of the invention. For example, the system 2500 may be used as part of the controller. Note that while FIG. 25 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 25, the computer system 2500, which is a form of a data processing system, includes a bus or interconnect 2502 which is coupled to one or more microprocessors 2503 and a ROM 2507, a volatile RAM 2505, and a non-volatile memory 2506. The microprocessor 2503 is coupled to cache memory 2504. The bus 2502 interconnects these various components together and also interconnects these components 2503, 2507, 2505, and 2506 to a display controller and display device 2508, as well as to input/output (I/O) devices 2510, which may be mice, keyboards, modems, network interfaces, printers, and other devices which are well-known in the art.

Typically, the input/output devices 2510 are coupled to the system through input/output controllers 2509. The volatile RAM 2505 is typically implemented as dynamic RAM

(DRAM) which requires power continuously in order to refresh or maintain the data in the memory. The non-volatile memory 2506 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 25 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 2502 may include one or more buses connected to each other through various bridges, controllers, and/or adapters, as is well-known in the art. In one embodiment, the I/O controller 2509 includes a USB (Universal Serial Bus) adapter for controlling USB peripherals. Alternatively, I/O controller 2509 may include IEEE-1394 adapter, also known as FireWire adapter, for controlling FireWire devices, SPI (serial peripheral interface), I2C (inter-integrated circuit) or UART (universal asynchronous receiver/transmitter), or any other suitable technology. Wireless communication protocols may include Wi-Fi, Bluetooth, ZigBee, near-field, cellular and other protocols.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the Figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the preceding Figs may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

From the foregoing, it will be appreciated that specific embodiments of the invention have been described herein for purposes of illustration, but that various modifications may be made without deviating from the spirit and scope of the various embodiments of the invention. For example, several embodiments may include various suitable combinations of components, devices and/or systems from any of the embodiments described herein. Further, while various advantages associated with certain embodiments of the invention have been described above in the context of those embodiments, other embodiments may also exhibit such advantages, and not all embodiments need necessarily exhibit such advantages to fall within the scope of the invention.

What is claimed is:

1. An apparatus for accessing a portion of a body comprising:
a cannula configured for insertion into the body;
a first electrode at or near a distal end of the cannula, wherein the first electrode is positioned to contact tissue adjacent to the distal end of the cannula during advancement into the body;
a second electrode;
a controller in communication with the first electrode via a first lead and in communication with the second electrode via a second lead;
where the first lead runs through an inner lumen of the cannula;
where the apparatus is configured to detect an impedance or a conductance of the tissue between the first electrode and the second electrode
wherein the controller is configured to receive one or more signals from the tissue adjacent to the distal end of the cannula and detect a change in property of the tissue as the cannula is advanced; and
wherein the controller is further configured to determine a position of the distal end of the cannula during advancement into the body.

2. The apparatus of claim 1 where the second electrode is positioned at or near the distal end of the cannula and the second lead runs through the inner lumen of the cannula.

3. The apparatus of claim 1 where the second electrode is positioned on a skin of the body.

4. The apparatus of claim 1 where the cannula includes a blunt distal tip.

5. The apparatus of claim 1 where the first lead is embedded in a wall of the cannula.

6. The apparatus of claim 1 where the first electrode is incorporated into a stylus that is configured to be inserted into the inner lumen of the cannula.

7. The apparatus of claim 6 where the second electrode is incorporated into the stylus.

8. The apparatus of claim 6 where the stylus is removable from the cannula.

9. The apparatus of claim 6 where the stylus includes a stylus inner lumen.

10. The apparatus of claim 6 which includes a locking mechanism to lock the stylus in place within the cannula.

11. The apparatus of claim 1 where the cannula is inserted into the body manually.

12. The apparatus of claim 1 where the cannula is inserted into the body automatically.

13. The apparatus of claim 1 where the first lead comprises an insulated portion of a shaft of the cannula and the first electrode comprises an uninsulated portion of the shaft of the cannula.

14. A method for accessing a portion of a body comprising;
advancing a cannula into the body so that the cannula contacts tissue adjacent to the cannula during advancement via a first electrode positioned at or near a distal end of the cannula;
positioning a second electrode so that it is in contact with the body;
receiving one or more signals from the tissue in contact with the first and second electrodes via the first lead and a second lead;
detecting a change in an impedance or a conductance of the tissue between the first electrode and the second electrode via a controller in communication with the first and second electrodes as the cannula is advanced;
determining, via the controller, a position of the cannula within the body based upon the change in the impedance or the conductance of the tissue between the first electrode and the second electrode as the cannula is advanced; and
where the first lead runs through an inner lumen of the cannula.

15. The method of claim 14 where the second electrode is positioned at or near the distal end of the cannula and the second lead runs through the inner lumen of the cannula.

16. The method of claim 14 where the cannula includes a blunt distal tip.

17. The method of claim 14 where the first electrode is incorporated into a stylus that is configured to be inserted into the inner lumen of the cannula.

18. The method of claim 17 where the second electrode is incorporated into the stylus.

19. The method of claim 14 where the first lead comprises an insulated portion of a shaft of the cannula and the first electrode comprises an uninsulated portion of the shaft of the cannula.

20. The method of claim 17 which includes a locking mechanism to lock the stylus in place within the cannula.
